(19) United States
(12) Patent Application Publication
(10) Pub. No.: US 2026/0365104 A1
(43) Pub. Date: Nov. 19, 2026

(54) LIGHTING SYSTEM

(71) Applicant: PANASONIC INTELLECTUAL PROPERTY MANAGEMENT CO., LTD., Osaka (JP)

(72) Inventor: Hikaru MAEDA, Osaka (JP)

(21) Appl. No.: 19/734,462

(22) Filed: Jul. 8, 2026

Related U.S. Application Data
(63) Continuation of application No. PCT/JP2024/043650, filed on Dec. 10, 2024.

(30) Foreign Application Priority Data
Jan. 9, 2024 (JP) 2024-001204

(51) Int. Cl. B60Q 3/53; B60Q 3/70; F21V 8/00

(52) U.S. Cl. CPC B60Q 3/53; B60Q 3/70; G02B 6/0046

(57) ABSTRACT

The lighting system includes at least one first lighting device that is installed in a ceiling in a vehicle interior of a moving body and emits awakening light, the at least one first lighting device being a long lighting device including at least one light source, and being disposed so as to at least partly overlap a movable range of a first seat provided in the vehicle interior when viewed from above.

### FIELD OF INVENTION

The present disclosure relates to a lighting system installed in a vehicle interior of a moving body.

### BACKGROUND ART

Patent Literature 1 discloses a vehicle interior lighting device including a light source attached to a recess disposed below a side end of a vehicle roof, the recess being a recess between a front seat assist grip and a rear seat assist grip. The light source is disposed to illuminate a vehicle interior after illumination light is reflected from a surface of the recess.

Patent Literature 2 discloses a vehicle interior lighting system in which a linear light emitting unit having a long linear light emitting portion extending in a longitudinal direction of a ceiling in a vehicle interior is provided at each of left and right edges of the ceiling in the vehicle interior, and light emitted by the linear light emitting portion directly illuminates the vehicle interior.

However, in the conventional technique described above, the lighting devices are provided at both left and right edges of the ceiling in the vehicle interior, and a purpose of the lighting devices is to illuminate the vehicle interior. Therefore, illuminance is insufficient for awakening an occupant in the seat. Therefore, the above-described conventional technique cannot awaken the occupant.
Patent Literature 1: JP4419990 B2
Patent Literature 2: JP4858364 B2

### SUMMARY OF THE INVENTION

The present disclosure has been made to solve such a problem, and an object of the present disclosure is to provide a lighting system that awakens an occupant in a seat.

A lighting system according to one aspect of the present disclosure includes at least one first lighting device that is installed in a ceiling in a vehicle interior of a moving body and emits awakening light, the at least one first lighting device is a long lighting device including at least one light source, and is disposed so as to at least partly overlap a movable range of a first seat provided in the vehicle interior when viewed from above.

According to the present disclosure, an occupant in a seat can be awakened.

### BRIEF DESCRIPTION OF THE DRAWINGS

FIG. 1 is a top view of a moving body in which a lighting system according to an embodiment of the present disclosure is disposed.

FIG. 2 is a view of the lighting system illustrated in FIG. 1 as viewed from a rear side to a front side of the moving body.

FIG. 3 is a view of a vehicle interior of the moving body as viewed from a left direction to a right direction.

FIG. 4 is a view illustrating a detailed configuration of a lighting device.

FIG. 5 is a view illustrating a detailed configuration of an edge type lighting device.

FIG. 6 is a view illustrating an attaching example of the lighting device.

FIG. 7 is a view illustrating another attaching example of the lighting device.

FIG. 8 is a view of the lighting system as viewed from the left direction to the right direction.

FIG. 9 is a view illustrating a detailed configuration of a lens type lighting device.

FIG. 10 is a view of a lighting system including one or more lighting devices provided at a position opposed to an occupant as viewed from the rear side to the front side of the moving body.

FIG. 11 is a view of the lighting system illustrated in FIG. 10 as viewed from the left direction to the right direction.

FIG. 12 is a view illustrating the lighting device illustrated in FIG. 11 in detail.

FIG. 13 is a block diagram showing an electrical configuration of the lighting system in the embodiment of the present disclosure.

FIG. 14 is a graph showing a change in illuminance of the lighting device in an awakening phase.

FIG. 15 is a view of a vehicle interior of a moving body as viewed from the left direction to the right direction in a first modification.

FIG. 16 is a view illustrating an attaching example of an acoustic device.

FIG. 17 is a view of a vehicle interior of a moving body as viewed from the left direction to the right direction in a second modification.

FIG. 18 is a view illustrating an attaching example of a sound pickup device.

FIG. 19 is a top view of a moving body in which an environment forming system is disposed according to a third modification.

### DETAILED DESCRIPTION

### Knowledge Underlying Present Disclosure

Techniques have been studied for controlling an environment inside a moving body using a plurality of apparatuses such as a display apparatus, an acoustic apparatus, and a lighting apparatus to guide an occupant of the moving body to an appropriate state. For example, during a traveling period from a departure point to an arrival at a destination, when an occupant is first guided to a relaxed state, then the occupant is guided to a sleep state, and finally the occupant is guided to an awake state, the occupant can be clearly awakened by the time of the arrival at the destination. As a result, the occupant can actively perform an action after arriving at the destination.

In order to awaken the occupant, it is necessary to irradiate the occupant with light having an illuminance of at least 100 lx (lux), and in an existing lighting device that illuminates the inside of a vehicle, the illuminance is insufficient and the occupant cannot be awakened.

Since in the vehicle interior lighting device of Patent Literature 1, the light source is disposed to illuminate the vehicle interior after reflecting the illumination light on the surface of the recess and does not directly irradiate an occupant, illuminance is insufficient to awaken the occupant. Although the vehicle interior lighting system of Patent Literature 2 directly illuminates the vehicle interior, the linear light emitting portions are provided on the left and right edges of the ceiling in the vehicle interior, and the purpose of the linear light emitting portions is to illuminate the vehicle interior, so that illuminance is insufficient to awaken the occupant.

Therefore, the present inventor has obtained knowledge that it is possible to awaken an occupant when a long lighting device is disposed so as to at least partly overlap a movable range of a seat of a moving body when viewed from above and the lighting device emits awakening light, and has conceived the present disclosure.
(1) A lighting system according to one aspect of the present disclosure includes at least one first lighting device that is installed in a ceiling in a vehicle interior of a moving body and emits awakening light, the at least one first lighting device is a long lighting device including at least one light source, and is disposed so as to at least partly overlap a movable range of a first seat provided in the vehicle interior when viewed from above.

According to this configuration, since at least one long first lighting device is disposed so as to at least partly overlap the movable range of the first seat as viewed from above, it is possible to irradiate an occupant with the awakening light from directly above and to awaken the occupant.
(2) In the lighting system according to (1), the at least one first lighting device may include a long light guide body, and the at least one light source may be provided to have a light emitting surface facing a longitudinal direction of the light guide body at one end portion in the longitudinal direction of the light guide body.

According to this configuration, since the light source is provided such that the light emitting surface is directed in the longitudinal direction of the long light guide body at one end portion of the light guide body, a heat dissipation member of the light source can be disposed in a front-rear direction in the vehicle interior. As a result, a height of the lighting device is shortened, and the lighting device can be attached so as not to protrude from a ceiling surface in the vehicle interior.
(3) The lighting system according to (1) or (2) may further include a second lighting device, the second lighting device being disposed to be opposed to a front of the first seat.

According to this configuration, since the occupant is irradiated with awakening light from the second lighting device from the front, the occupant can be more reliably awakened.
(4) In the lighting system according to any one of (1) to (3), the first seat may be a rear seat, and the second lighting device may be installed between a front seat and the rear seat.

According to this configuration, since a front of the rear seat can be irradiated with the awakening light, the occupant in the rear seat can be more reliably awakened.
(5) In the lighting system according to any one of (1) to (4), the at least one first lighting device may have variable illuminance, and the lighting system may further include a control device that changes the illuminance of the at least one first lighting device over time in an awakening phase of awakening an occupant.

According to this configuration, in the awakening phase, the illuminance of the awakening light output from the at least one first lighting device can be changed over time to awaken the occupant in a more comfortable state.
(6) In the lighting system according to (3), the second lighting device may have variable illuminance, and the lighting system may further include a control device that changes the illuminance of the second lighting device over time.

According to this configuration, the illuminance of the awakening light output from the second lighting device can be changed over time to awaken the occupant in a more comfortable state.
(7) In the lighting system according to any one of (1) to (6), the ceiling may include an attachment port to which each of the at least one first lighting device is attached and a reinforcing member provided along the attachment port on a back surface of the ceiling, each of the at least one first lighting device may include: a housing embedded in the attachment port; and a first cantilever spring connected to one side surface in a longitudinal direction of the housing and a second cantilever spring connected to the other side surface in the longitudinal direction of the housing, and free ends of the first and second cantilever springs may be brought into contact with the reinforcing member.

According to this configuration, when the first lighting device is inserted into the attachment port, the free end of the first cantilever spring and the free end of the second cantilever spring are opened so as to be separated from the side surface in the longitudinal direction of the housing, and contact the reinforcing member provided along the attachment port on the back surface of the ceiling, so that the first lighting device is fixed inside the ceiling. Therefore, the first lighting device can be attached without being screwed to the ceiling, and designability in the vehicle interior can be enhanced. In addition, it is possible to prevent a body of the occupant from colliding with the first lighting device, and it is possible to enhance safety of the occupant in the vehicle interior. In addition, since the free end contacts the reinforcing member and does not directly contact the ceiling, the ceiling can be protected.
(8) In the lighting system according to any one of (1) to (7), in the at least one first lighting device, luminance of the at least one light source may be set such that illuminance with respect to a front surface of a headrest portion of the first seat is at least 100 lx

In order to awaken a person, it is necessary to emit at least 100 lx of light. In this configuration, since the first lighting device is configured such that the illuminance with respect to the front surface of the headrest portion of the first seat is at least 100 lx, the occupant can be reliably awakened.
(9) In the lighting system according to (5), the control device may increase the illuminance of the at least one first lighting device over time from a turn-off state to a maximum illuminance in the awakening phase.

According to this configuration, since in the first lighting device, the illuminance is increased over time from the turn-off state to the maximum illuminance, the occupant can be naturally awakened.
(10) The lighting system according to any one of (1) to (9) may further include at least one third lighting device, the at least one third lighting device being an indirect lighting device installed between a front seat and a rear seat.

According to this configuration, since the third lighting device is provided, the illuminance of the awakening light with which the occupant is irradiated can be further increased, and the occupant can be more reliably awakened. Furthermore, since the third lighting device is an indirect lighting, it is possible to enhance the designability of the vehicle interior.
(11) The lighting system according to any one of (1) to (10) may further include an acoustic device installed in the ceiling, the acoustic device being disposed so as to at least partly overlap the movable range of the first seat when viewed from above.

According to this configuration, hearing of the occupant can be stimulated to awaken the occupant.
(12) The lighting system according to any one of (1) to (11) may further include a sound pickup device installed in the ceiling, the sound pickup device being disposed so as to at least partly overlap the movable range of the first seat when viewed from above.

According to this configuration, sound of the occupant can be picked up to check a state of the occupant.
(13) An environment forming system according to another aspect of the present disclosure includes an acoustic device installed in a ceiling in a vehicle interior of a moving body, in which the acoustic device is disposed so as to at least partly overlap a movable range of a first seat provided in the vehicle interior when viewed from above.
(14) An environment forming system according to a further aspect of the present disclosure includes a sound pickup device installed in the ceiling in the vehicle interior of the moving body, in which the sound pickup device is disposed so as to at least partly overlap the movable range of the first seat provided in the vehicle interior when viewed from above.

Note that each embodiment to be described below illustrates a specific example of the present disclosure. Numerical values, shapes, components, steps, orders of steps, and the like of embodiments below are merely examples, and are not intended to limit the present disclosure. A component not described in an independent claim representing a highest concept among components in the embodiments below is described as an optional component. In all the embodiments, each content can be combined.

### EMBODIMENTS

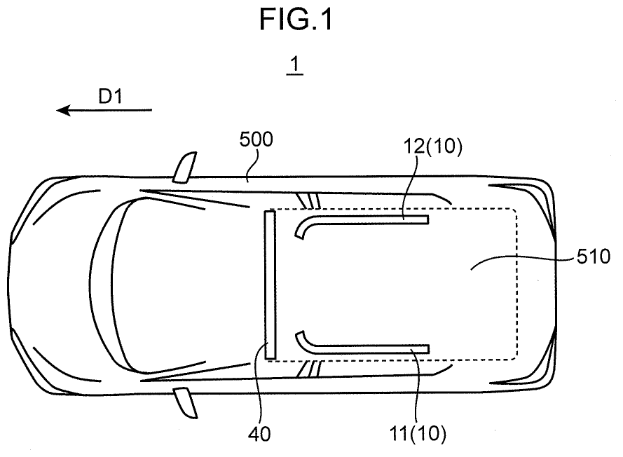

FIG. 1 is a top view of a moving body 500 in which a lighting system 1 according to an embodiment of the present disclosure is disposed. The lighting system 1 is disposed in a vehicle interior of the moving body 500. The lighting system 1 includes a lighting device 10 (an example of a first lighting device) and a display device 40. The lighting device 10 is provided in a ceiling 510 in the vehicle interior. The lighting device 10 includes a pair of a lighting device 11 and a lighting device 12. The lighting devices 11 and 12 each include at least one light source and are long in a traveling direction D1 of the moving body 500. The lighting devices 11 and 12 are disposed on the ceiling 510 in the vehicle interior of the moving body 500.

The moving body 500 includes a front seat on a front side in the traveling direction D1 in the vehicle interior and a rear seat (an example of a first seat) on a rear side. The lighting devices 11 and 12 are disposed so as to at least partly overlap a movable range of the rear seat when viewed from above the moving body. The lighting devices 11 and 12 are parallel and have the same length. Distal ends of the lighting devices 11 and 12 are curved toward the center of the moving body 500. The display device 40 is provided between the front seat and the rear seat. The display device 40 presents video contents to an occupant in the rear seat.

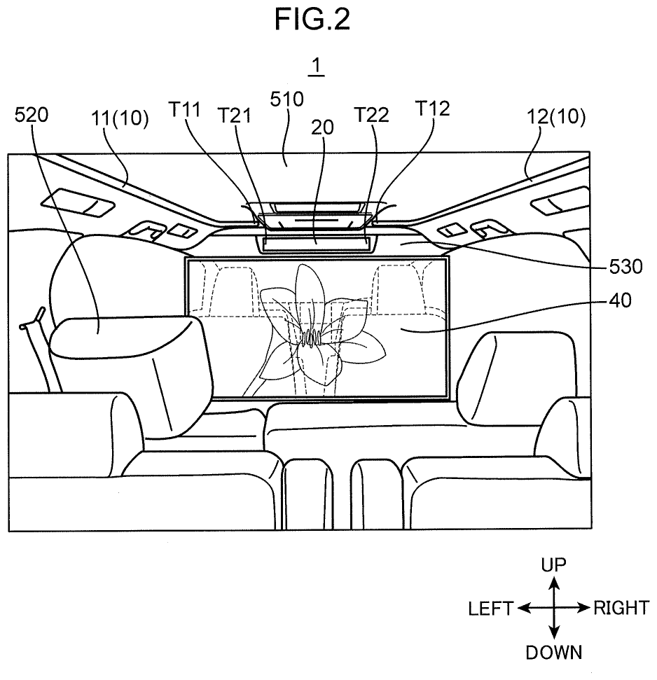

FIG. 2 is a view of the lighting system 1 illustrated in FIG. 1 as viewed from a rear side to a front side of the moving body 500. The lighting system 1 further includes a lighting device 20 (an example of a second lighting device). The lighting device 20 and the display device 40 are installed on a wall surface 530 provided between the front seat and a rear seat 520. The rear seat 520 is a seat on which three persons can sit side by side in a left-right direction of the moving body 500. The left-right direction is a direction parallel to a floor of the moving body 500 and orthogonal to a front-rear direction. A left direction refers to a left side direction when the moving body 500 is viewed from the rear side to the front side, and the right direction refers to a right side direction when the moving body 500 is viewed from the rear side to the front side. The lighting device 20 is installed above the display device 40. The lighting device 20 is long in the left-right direction. The display device 40 is a large-screen display device that occupies most of the wall surface 530. A display surface of the display device 40 straightly faces the rear seat 520.

The lighting device 11 has a distal end T11 curved toward a center side of the moving body 500. The lighting device 12 has a distal end T12 curved toward the center side of the moving body 500. When viewed from above, the distal end T11 and a distal end T21 of the lighting device 20 are substantially continuous. When viewed from above, the distal end T12 and a distal end T22 of the lighting device 20 are substantially continuous. Although in this example, the distal ends T11 and T21 are curved toward the center, they may not be curved. In this case, the lighting devices 11 and 12 are linear.

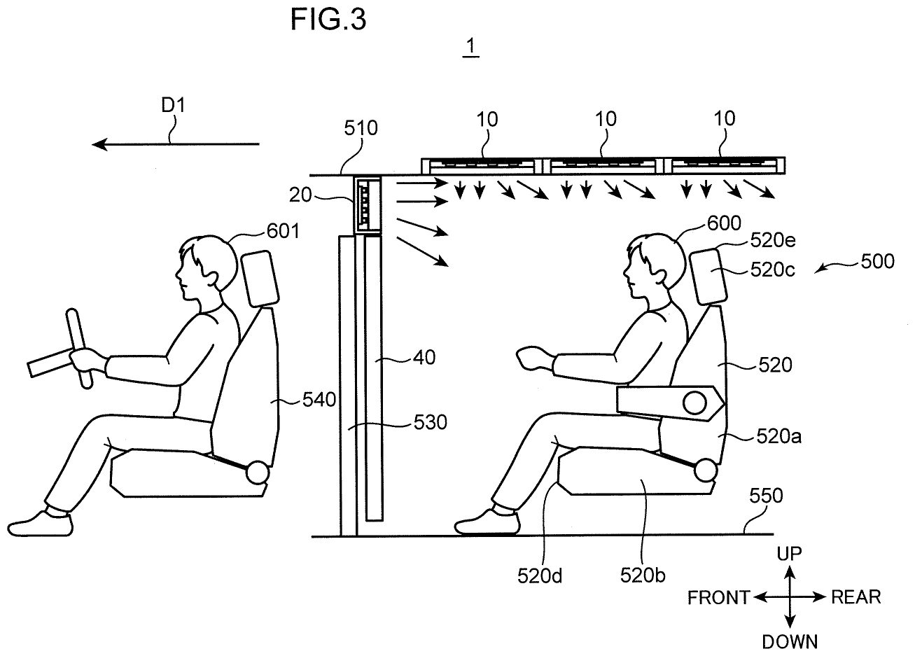

FIG. 3 is a view of the vehicle interior of the moving body 500 as viewed from the left direction to the right direction. The moving body 500 includes the ceiling 510, the rear seat 520, the wall surface 530, a front seat 540, and a floor 550. A driver 601 is seated on the front seat 540. The driver 601 is an occupant who operates the moving body 500. The occupant 600 is seated on the rear seat 520. The occupant 600 is an occupant who is seated on the rear seat 520. The wall surface 530 is erected in an up-down direction with respect to the floor 550. The lighting device 20 is provided between the ceiling 510 and the display device 40.

The lighting device 20 (an example of the second lighting device) is disposed such that a light emitting surface is opposed to a front of the rear seat 520. As a result, the lighting device 20 can irradiate the occupant 600 from the front. In addition, since the light emitting surface of the lighting device 20 faces backward and does not face forward, it is possible to prevent an awakening light from the lighting device 20 from being reflected on a windshield and disturbing driving of the driver.

The display device 40 is configured by a transparent display. The wall surface 530 has an opening in a region where the display device 40 is located. Therefore, scenery in front of the moving body 500 passes through the wall surface 530 and the display device 40. This enables the occupant 600 to visually recognize both the scenery in front and an image displayed on the display device 40. The display device 40 is not limited to a transparent display, and may be a display of a smartphone or a display of a tablet computer in addition to a normal display. Furthermore, the display device 40 may be a screen for projecting video contents.

In the example of FIG. 3, three lighting devices 10 are disposed in a line in the traveling direction D1. The number of the lighting devices 10 disposed in a line is not limited to three, and may be one, two, or four or more. The traveling direction D1 is the same as the front-rear direction. Note that among the three lighting devices 10, only the lighting device 10 immediately above the position where the occupant 600 is seated may be turned on, and the remaining lighting devices may be turned off. This enables saving of power consumption.

The rear seat 520 includes a reclinable back portion 520a, a seat portion 520b on which buttocks of the occupant 600 are placed, and a headrest 520c provided on an upper side of the back portion 520a. The rear seat 520 is configured to be slidable in the front-rear direction. The movable range of the rear seat 520 refers to a section in the front-rear direction from a position of a distal end 520d of the seat portion 520b when the rear seat 520 is slid to the foremost side and the back portion 520a is raised most to a position of a distal end 520e of the headrest 520c when the rear seat 520 is slid to the rearmost side and the back portion 520a is inclined to the rearmost side.

In the example of FIG. 3, the lighting device 10 is disposed so as to overlap substantially the entire movable range of the rear seat 520 when viewed from above. Note that this is merely an example, and the lighting device 10 only needs to be disposed so as to overlap at least the rear seat 520 when viewed from above in a case where a slide position of the rear seat 520 is at a normal position and a degree of inclination of the back portion 520a is normal.

By providing the lighting device 10 in the ceiling 510, it is easy to irradiate a face of the occupant 600 with an awakening light having illuminance of at least 100 lx regardless of a degree of reclining of the rear seat 520.

The lighting device 10 is configured such that a direction of light distribution varies depending on a position in the front-rear direction. In this example, the lighting device 10 is configured such that the light distribution faces directly downward on the front side, and a degree of inclination of backward light distribution increases toward the rear side. In addition, the lighting device 10 has luminance and the light distribution set such that the illuminance of the awakening light at the headrest 520c becomes at least 100 lx. As a result, the face of the occupant 600 is irradiated with the awakening light of at least 100 lx, and the occupant 600 can be reliably awakened. Note that the luminance of the lighting device 10 may be set such that face illuminance of the occupant 600 is at least 100 lx even for the occupant 600 wearing AR glasses.

The lighting device 20 is configured such that a direction of light distribution varies depending on a position in the up-down direction. In this example, the lighting device 20 is configured such that the light distribution faces directly backward on the upper side, and a degree of inclination of downward light distribution increases toward the lower side. This enables the lighting device 10 and the lighting device 20 to irradiate the face of the occupant 600 with the awakening light regardless of the slide position and the reclining state of the rear seat 520 and a body shape of the occupant 600. As a result, the occupant 600 can be reliably awakened. In addition, the lighting device 20 has luminance and the light distribution set such that the illuminance of the awakening light at the headrest 520c becomes at least 100 lx. As a result, the face of the occupant 600 is irradiated with the awakening light of at least 100 lx, and the occupant 600 can be reliably awakened. Note that the luminance of the lighting device 20 is set such that the face illuminance of the occupant 600 is at least 100 lx even for the occupant 600 wearing the AR glasses.

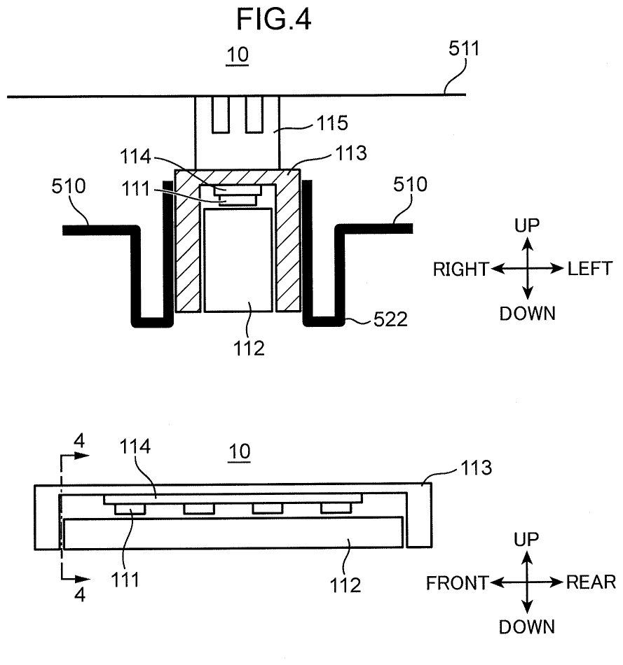

FIG. 4 is a view illustrating a detailed configuration of the lighting device 10. An upper view is a cross-sectional view of the lighting device 10 from a direction 4-4 in a lower view, and the lower view is a view of the lighting device 10 viewed from the left direction to the right direction. In FIG. 4, a substrate mounting type lighting device 10 is illustrated. The lighting device 10 includes a light source 111, a light guide body 112, a housing 113, a substrate 114, and a heat dissipation member 115. The housing 113 has a prismatic shape with an opened lower portion. The substrate 114 is installed on an upper wall inside the housing 113. A plurality of the light sources 111 are disposed in a line in the front-rear direction on a lower surface of the substrate 114. The light source 111 is configured by, for example, a light emitting diode. The light source 111 has a light emitting surface facing downward. The light guide body 112 is provided below the light source 111 inside the housing 113. The light guide body 112 has a prismatic shape long in the front-rear direction. The light guide body 112 is made of polycarbonate or acrylic resin.

The heat dissipation member 115 is provided between an outer upper surface of the housing 113 and an outer wall 511 of the moving body 500. The heat dissipation member 115 cools the light source 111. The light distribution of the lighting device 10 can be adjusted by performing prism processing on the light guide body 112.

Since in the substrate mounting type lighting device 10, a back surface of the light source 111 faces upward, it is necessary to dispose the heat dissipation member 115 on the outer upper surface of the housing 113. As a result, a height of the lighting device 10 becomes larger than a height from the outer wall 511 to the ceiling 510 of the moving body 500, and the lighting device 10 protrudes downward from the ceiling 510. In this case, designability of the vehicle interior is impaired, and a body of the occupant 600 may collide with the lighting device 10.

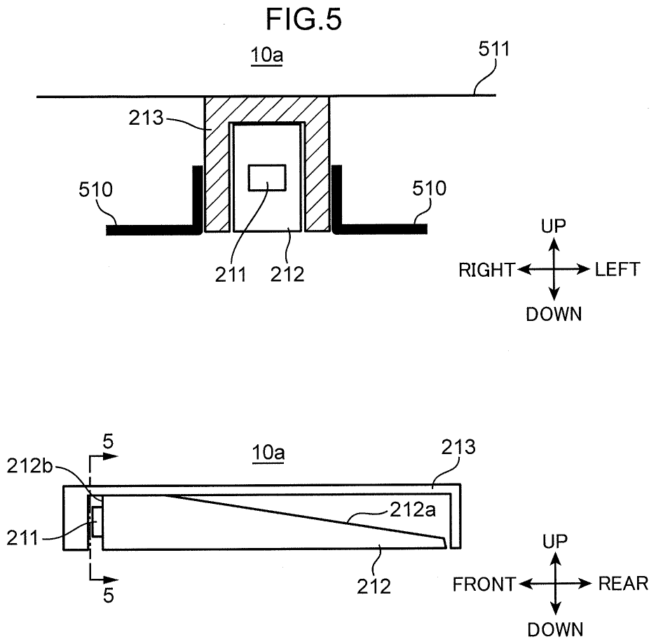

Therefore, in the present embodiment, it is also possible to adopt the following edge type lighting device 10a instead of the substrate mounting type lighting device 10. FIG. 5 is a view illustrating a detailed configuration of an edge type lighting device 10a. In FIG. 5, an upper view is a cross-sectional view of the lighting device 10a from a direction 5-5 illustrated in a lower view, and the lower view is a view of the lighting device 10a viewed from the left direction to the right direction.

The lighting device 10a includes a light source 211, a light guide body 212, and a housing 213. The housing 213 has a prismatic shape with an opened lower portion. The light guide body 212 is disposed inside the housing 213. The light guide body 212 is made of polycarbonate or acrylic resin. The light guide body 212 is long in the front-rear direction. The light guide body 212 has a tapered portion 212a having a width in the up-down direction shortened toward the rear side. A lower surface of the light guide body 212 is parallel to the floor 550. As a result, the light guide body 212 can direct light emitted backward from the light source 211 downward.

The light source 211 is provided at a front side end portion 212b of the light guide body 212. The light source 211 is configured by, for example, a light emitting diode. A light emitting surface of the light source 211 faces backward. In the lighting device 10a, since the light source 211 is provided at the end portion 212b, a heat dissipation member (not illustrated) can be disposed on a front side surface of the housing 213. This makes a height of the lighting device 10a be equal to or less than the height from the outer wall 511 to the ceiling 510, thereby preventing the lighting device 10a from protruding downward from the ceiling 510. As a result, it is possible to ensure the designability of the vehicle interior and to prevent the body of the occupant 600 from colliding with the lighting device 10a. Light distribution of the lighting device 10a can be adjusted by performing prism processing on the light guide body 212.

Consideration will be given to a mode in which the light source 211 is disposed on the rear side of the lighting device 10a and the light source 211 is disposed such that the light emitting surface of the light source 211 faces the front side. In this mode, since awakening light from the light source 211 is directed forward, there is a possibility that the awakening light leaks from the display device 40 and the wall surface 530 to the front seat 540 side, and is reflected on the windshield to disturb driving of the driver. In the example of FIG. 5, since the light source 211 is provided on the front side so that the light emitting surface faces backward, it is possible to prevent the awakening light from leaking to the front seat 540 side and being reflected on the windshield.

In the example of FIG. 3, the lighting device 20 may be configured by the edge type lighting device 10a. In a case where the lighting device 20 is configured by the edge type lighting device 10a, the housing 213 is disposed such that a longitudinal direction thereof faces the left-right direction. The end portion 212b of the light guide body 212 may be disposed on the left side or the right side. In a case where the end portion 212b is disposed on the left side, the light source 211 is disposed on the left side such that the light emitting surface faces the center, and in a case where the end portion 212b is disposed on the right side, the light source 211 is disposed on the right side such that the light emitting surface faces the center.

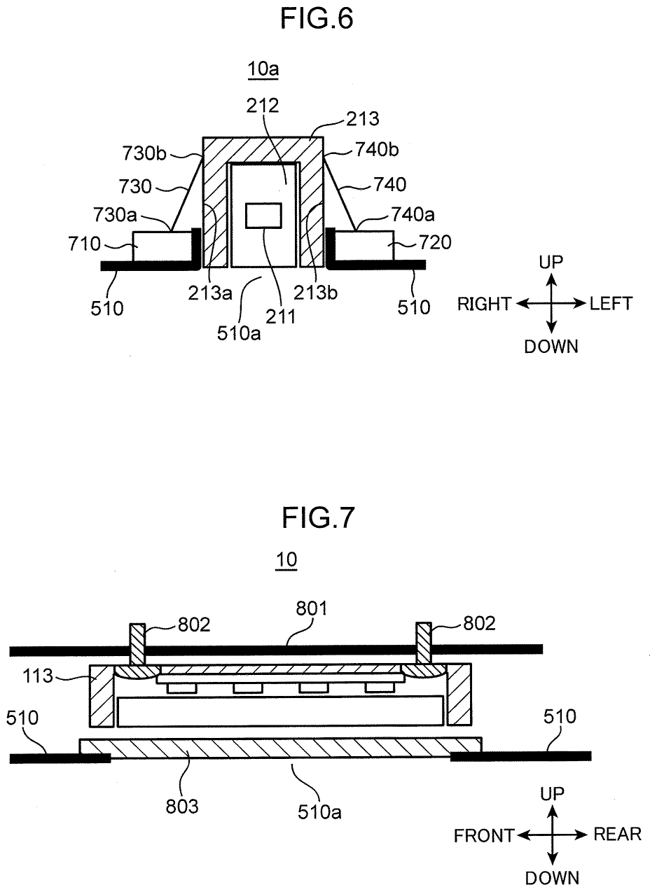

FIG. 6 is a view illustrating an attaching example of the lighting device 10a. In the example of FIG. 6, the lighting device 10a viewed from the front is illustrated. The ceiling 510 includes an attachment port 510a to which the lighting device 10a is attached. On a back surface of the ceiling 510, a pair of reinforcing members 710 and 720 provided along the attachment port 510a is provided. The attachment port 510a has a rectangular shape long in the front-rear direction and having substantially the same shape as a lower surface of the lighting device 10a. The reinforcing member 710 is provided along one side in a longitudinal direction of the attachment port 510a on the back side of the ceiling 510. The reinforcing member 720 is provided along the other side in the longitudinal direction of the attachment port 510a on the back side of the ceiling 510. The reinforcing members 710 and 720 are made of resin. The reinforcing members 710 and 720 have a prismatic shape long in the front-rear direction.

The lighting device 10a further includes a pair of cantilever springs 730 and 740 (an example of first and second cantilever springs). The housing 213 is embedded in the attachment port 510a. The cantilever springs 730 and 740 are plate-shaped elastic bodies. The cantilever spring 730 is connected to one side surface 213a in the longitudinal direction of the housing 213. The cantilever spring 740 is connected to the other side surface 213b in the longitudinal direction of the housing 213. The cantilever spring 730 includes a fixed end 730b connected to the side surface 213a and a free end 730a. The cantilever spring 740 includes a fixed end 740b connected to the side surface 213b and a free end 740a. The free end 730a is brought into contact with an upper surface of the reinforcing member 710. The free end 740a is brought into contact with an upper surface of the reinforcing member 720. The cantilever spring 730 applies tension to the side surface 213a and to the reinforcing member 710. The cantilever spring 740 applies tension to the side surface 213b and to the reinforcing member 720. As a result, the lighting device 10a is held in the ceiling 510 by the cantilever springs 730 and 740.

At the time of attachment, the lighting device 10a is inserted upward from below the attachment port 510a. When the lighting device 10a is inserted deep into the attachment port 510a, the free end 730a of the cantilever spring 730 and the free end 740a of the cantilever spring 740 are opened so as to be separated from the side surfaces 213a and 213b of the housing 213, respectively. This brings the free end 730a into contact with the upper surface of the reinforcing member 710 and the free end 740a into contact with the upper surface of the reinforcing member 720. As a result, the lighting device 10a is fixed inside the ceiling 510. Therefore, the lighting device 10a can be attached without being screwed to the ceiling 510, and the designability in the vehicle interior can be enhanced. In addition, since the free ends 730a and 740a contact the reinforcing members 710 and 720, respectively, and do not directly contact the ceiling 510, deformation of the ceiling 510 is prevented, and the ceiling 510 can be protected. In addition, since deformation of the ceiling 510 is prevented, it is possible to prevent the lighting device 10a from falling from the ceiling 510. Note that the cantilever springs 730 and 740 in FIG. 6 may be applied to the substrate mounting type lighting device 10.

FIG. 7 is a view illustrating another attaching example of the lighting device 10. The moving body 500 includes a scroll attachment plate 801. The scroll attachment plate 801 is a plate provided inside the ceiling 510 and parallel to the ceiling 510. The lighting device 10 is fastened to the scroll attachment plate 801 with a plurality of screws 802. The screws 802 are fastened to the scroll attachment plate 801 via the upper surface of the housing 113. In this example, a diffusion material 803 is attached to the attachment port 510a. The diffusion material 803 is made of milky white polycarbonate. The diffusion material 803 is disposed so as to be continuous with the ceiling 510. The awakening light emitted from the lighting device 10 is led out into the vehicle interior through the diffusion material 803.

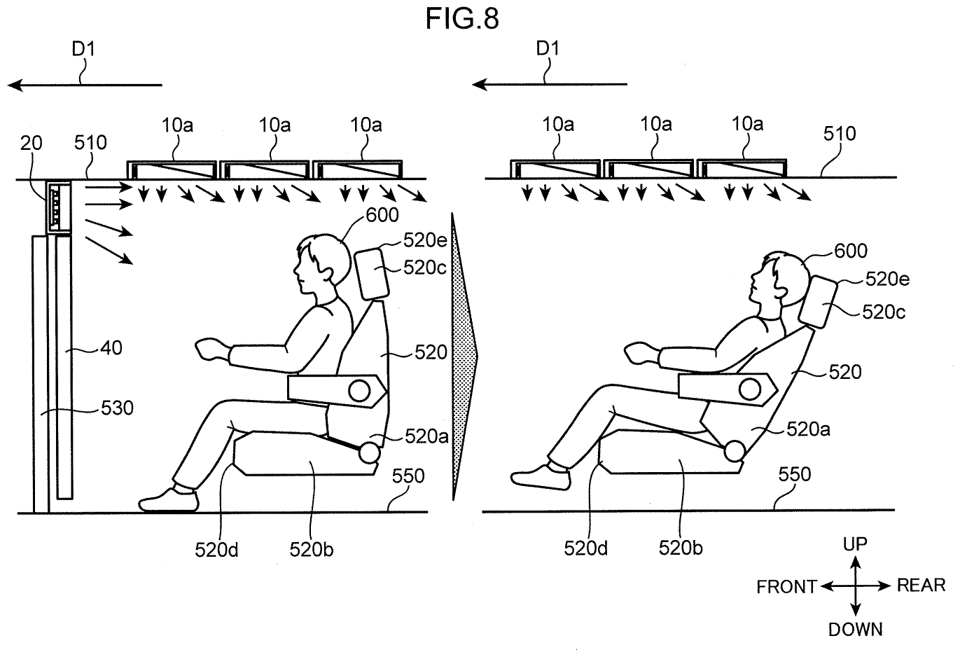

FIG. 8 is a view of the lighting system 1 as viewed from the left direction to the right direction, in which a left view illustrates a case where the rear seat 520 is at the slide position of the normal position, and a right view illustrates a case where the rear seat 520 is at the rearmost slide position. In the left view, the degree of reclining of the back portion 520a is normal. In the right view, the degree of reclining of the back portion 520a is maximum.

In the example of FIG. 8, three edge type lighting devices 10a are disposed in a line in the front-rear direction. Similarly to the lighting device 10, the lighting device 10a is configured such that the light distribution faces directly downward on the front side, and a degree of inclination of backward light distribution increases toward the rear side. As a result, the lighting device 10a can prevent the awakening light from reflecting on the windshield and irradiate the face of the occupant 600 with the awakening light.

In a case where a state of the rear seat 520 is the normal state illustrated in the left view of FIG. 8, the light distribution of the lighting devices 10a and 20 is set such that the face of the occupant is irradiated with the awakening light. In addition, also when the rear seat 520 is in the maximum reclining state illustrated in the right view of FIG. 8, the light distribution of the lighting devices 10a and 20 is set such that the face of the occupant is irradiated with the awakening light. As a result, in a case where the state of the rear seat 520 is at least within a range from the normal state to the maximum reclining state, the lighting devices 10a and 20 can reliably irradiate the face of the occupant with the awakening light.

Note that the face illuminance of the awakening light obtained by combining all the lighting devices 10a and 20 may be 100 lx. Furthermore, for example, even when the awakening light of the lighting device 20 does not hit the face of the occupant in the reclined state, the awakening light having the face illuminance of 100 lx may be emitted only by the lighting device 10a.

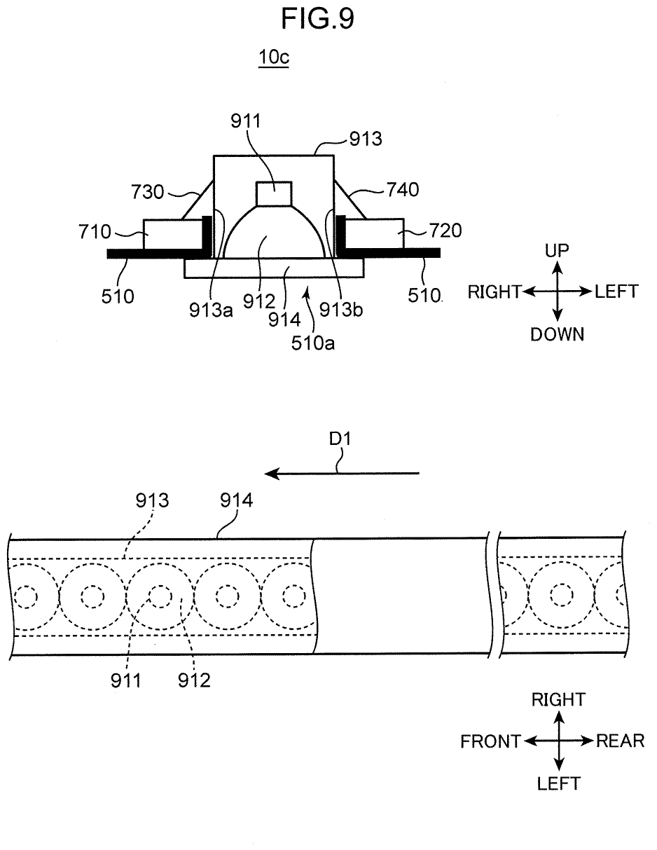

In the present embodiment, a lens type lighting device 10c illustrated in FIG. 9 may be adopted instead of the substrate mounting type lighting device 10 and the edge type lighting device 10a.

FIG. 9 is a view illustrating a detailed configuration of the lens type lighting device 10c. In FIG. 9, a left view is a view of the lighting device 10c viewed from the front side, and a right view is a view of the lighting device 10c viewed from the lower side. The lighting device 10c includes a light source 911, a light guide body 912, and a housing 913. A plurality of the light sources 911 are disposed in a line along the front-rear direction. The light source 911 is, for example, a light emitting diode. A plurality of the light guide bodies 912 are disposed corresponding to the plurality of light sources 911, respectively. The light guide body 912 is made of, for example, polycarbonate or acrylic resin. An upper surface of the light guide body 912 is connected to a light emitting surface of the light source 911. The light guide body 912 has a dome shape in which an area of a circular cross section increases toward the lower side, and functions as a lens. The light guide body 912 has a lower surface continuous with the attachment port 510a. The housing 913 has an opened lower surface and has a long prismatic shape along the traveling direction D1. A frame body 914 is provided below the housing 913 so as to be exposed from the ceiling 510. An awakening light from the light source 911 is led out into the vehicle interior through the light guide body 912 and an opening of the frame body 914.

The cantilever springs 730 and 740 described with reference to FIG. 6 are attached to side surfaces 913a and 913b of the housing 913. This causes the lighting device 10c to be inserted into the ceiling 510 from the attachment port 510a and held in the ceiling 510 by the tension of the cantilever springs 730 and 740. Note that the lighting device 20 may be configured by a lens type lighting device 10c. In this case, the housing 913 is disposed so as to have a longitudinal direction thereof facing the left-right direction.

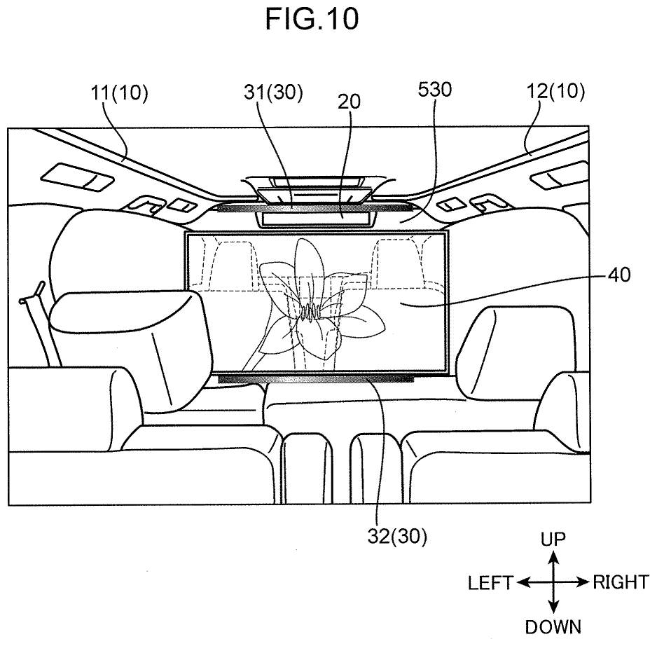

The lighting system 1 may include one or more lighting devices at a position opposed to the occupant 600. FIG. 10 is a view of the lighting system 1 including one or more lighting devices provided at a position opposed to the occupant 600 as viewed from the rear side to the front side of the moving body 500.

The lighting system 1 further includes a lighting device 31 provided above the display device 40 and a lighting device 32 provided below the display device 40. The lighting devices 31 and 32 are collectively referred to as a lighting device 30. The lighting device 30 is an example of a third lighting device.

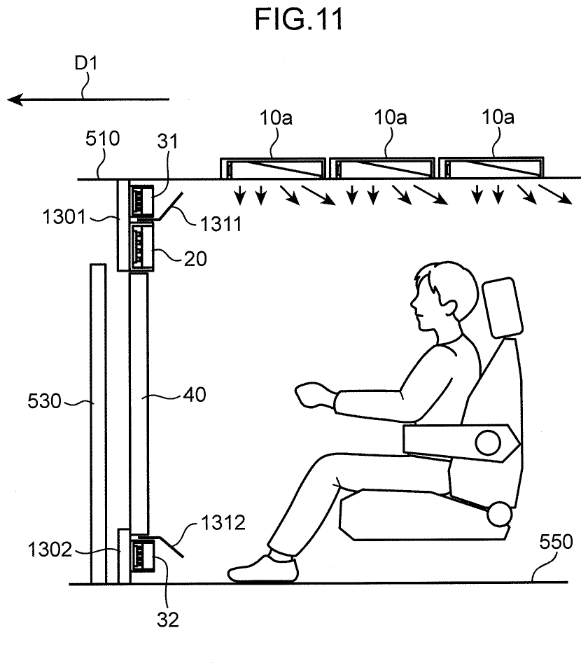

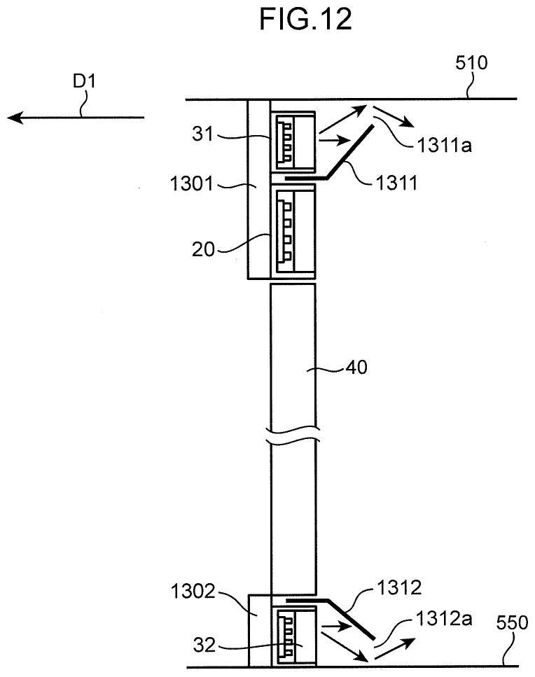

FIG. 11 is a view of the lighting system 1 illustrated in FIG. 10 as viewed from the left direction to the right direction. FIG. 12 is a view illustrating in detail the lighting devices 31 and 32 illustrated in FIG. 11. The lighting device 31 is attached to a wall surface 1301 extending downward from the ceiling 510.

The lighting device 32 is attached to a wall surface 1302 extending upward from the floor 550. Similarly to the lighting device 10, the lighting devices 31 and 32 are configured by substrate mounting type lighting devices. Note that this is an example, and the lighting devices 31 and 32 may be configured by an edge type lighting device 10a or the lens type lighting device 10c. The lighting devices 31 and 20 are attached to the wall surface 1301 such that light emitting surfaces of the lighting devices 31 and 20 are continuous with the display surface of the display device 40. The lighting device 32 is attached to the wall surface 1302 such that a light emitting surface of the lighting device 32 is continuous with the display surface of the display device 40. Consequently, an awakening light can be prevented from being reflected on the display device 40. In the example of FIG. 11, the edge type lighting device 10a is attached to the ceiling 510. Note that this is an example, and the substrate mounting type lighting device 10 may be attached to the ceiling 510, or the lens type lighting device 10c may be attached to the same.

Refer to FIG. 12. A fixture member 1311 that converts an awakening light from the lighting device 31 into an indirect illumination light is provided below the lighting device 31. The fixture member 1311 has a shape inclined so that a distance from the light emitting surface of the lighting device 31 gradually increases toward the upper side, and is attached below the lighting device 31. The fixture member 1311 is configured such that a gap 1311a is formed between an upper end of the fixture member 1311 and the ceiling 510. This causes the awakening light from the lighting device 31 to be reflected by the fixture member 1311 and then led out into the vehicle interior through the gap 1311a.

A fixture member 1312 that converts an awakening light from the lighting device 32 into an indirect illumination light is provided above the lighting device 32. The fixture member 1312 has a shape inclined so that a distance from the light emitting surface of the lighting device 32 gradually increases toward the lower side, and is attached above the lighting device 32. The fixture member 1312 is configured such that a gap 1312a is formed between a lower end of the fixture member 1312 and the floor 550. This causes the awakening light from the lighting device 32 to be reflected by the fixture member 1312 and then led out into the vehicle interior through the gap 1312a. As a result, since the indirect illumination light is led out into the vehicle interior as the awakening light, it is possible to prevent the awakening lights from the lighting devices 31 and 32 from directly entering an eye of the occupant 600.

The lighting devices 31 and 32 include light sources of four colors of red (R), green (G), blue (B), and white (W) disposed in a matrix. Thus, lights of four colors are mixed in a space between the fixture member 1311 and the lighting device 31 and a space between the fixture member 1312 and the lighting device 32, and color unevenness of the awakening light can be prevented. In addition, since the light emitting surfaces of the lighting devices 31 and 32 and the lighting device 20 face the occupant 600 side, attachment of the lighting devices 31, 32, and 20 is facilitated.

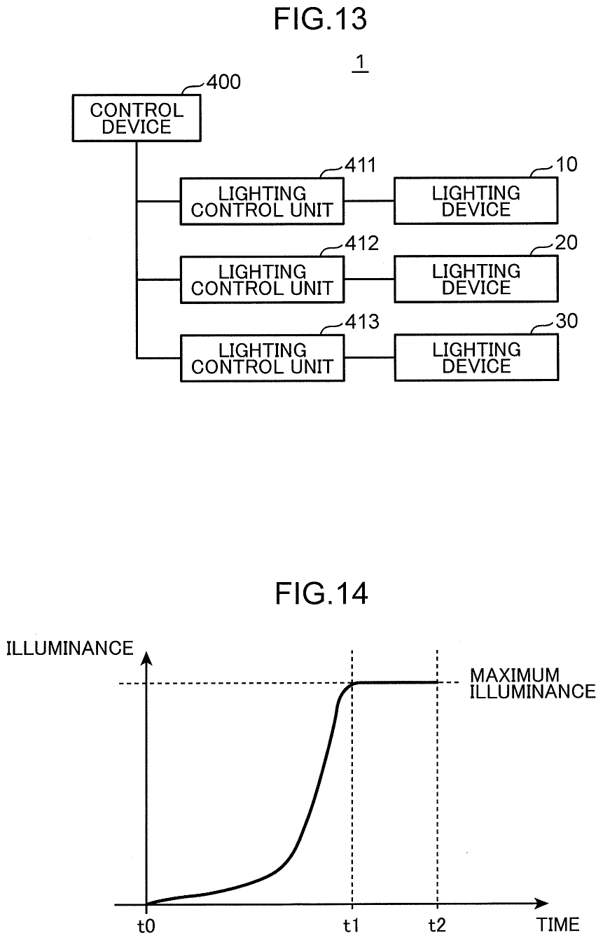

FIG. 13 is a block diagram showing an electrical configuration of the lighting system 1 in the embodiment of the present disclosure. The lighting system 1 includes a control device 400, lighting control units 411, 412, and 413, and the lighting devices 10, 20, and 30.

The control device 400 is configured by, for example, a computer including a central processing unit (CPU) and a memory. The control device 400 outputs a control signal to the lighting control units 411 to 413 to control illuminance of the lighting devices 10, 20, and 30.

The lighting control unit 411 is configured by a drive circuit that controls the lighting device 10, and adjusts the illuminance of the lighting device 10 according to the control signal output from the control device 400. The lighting control unit 412 is configured by a drive circuit that controls the lighting device 20, and adjusts the illuminance of the lighting device 20 according to the control signal output from the control device 400. The lighting control unit 413 is configured by a drive circuit that controls the lighting device 30, and adjusts the illuminance of the lighting device 30 according to the control signal output from the control device 400.

The control device 400 may cause the display device 40 to display video contents so as to guide the occupant 600 to a state corresponding to a plurality of phases, may cause an acoustic device (not illustrated) to output acoustic contents, may cause the lighting devices 10 to 30 to perform dimming, or may cause a diffuser (not illustrated) to output a scent.

As the plurality of phases, a relaxation phase, a sleep phase subsequent to the relaxation phase, and an awakening phase subsequent to the sleep phase can be adopted. The relaxation phase is a phase for guiding the occupant 600 to a relaxed state. The sleep phase is a phase for guiding the occupant 600 to a sleep state. The awakening phase is a phase for guiding the occupant 600 to an awake state. By performing the relaxation phase and the sleep phase in this order, it is possible to cause the occupant 600 to securely sleep. In addition, by performing the sleep phase and the awakening phase in this order, it is possible to cause the occupant 600 to be awakened in a clear state. As a result, the occupant 600 can actively perform an action after arriving at a destination.

The control device 400 controls operations of the lighting devices 10 to 30, the display device 40, the acoustic device, and the diffuser according to the plurality of phases. For example, the control device 400 may decide contents in the relaxation phase, contents in the sleep phase, and contents in the awakening phase from a departure point to the destination, and output a control signal for reproducing the decided contents to the display device 40 and the acoustic device.

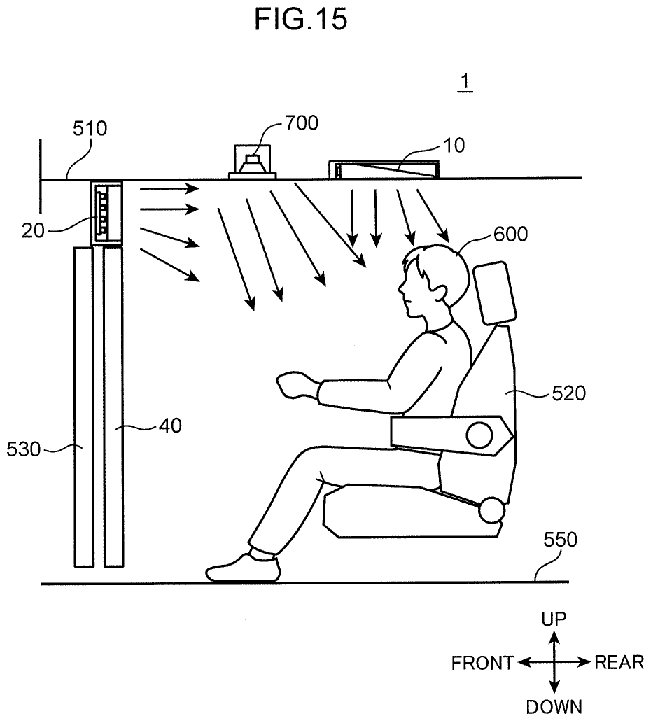

FIG. 14 is a graph showing a change in illuminance of the lighting devices 10 to 30 in the awakening phase. In FIG. 14, the vertical axis represents illuminance, and the horizontal axis represents time. Time t0 is start time of the awakening phase. Time t2 is end time of the awakening phase. When the awakening phase is started, the control device 400 outputs, to the lighting control units 411 to 413, a control signal for increasing the illuminance of the lighting devices 10 to 30 over time from zero (turn-off state) to a maximum illuminance. As a result, the illuminance of the awakening light output by the lighting devices 10 to 30 increases over time from zero to the maximum illuminance. Here, the control device 400 may increase the illuminance of the awakening light in a quadratic function manner or linearly. In the example of FIG. 14, the illuminance is increased in a quadratic function manner. By increasing the illuminance over time, it is possible to prevent the occupant 600 from awaking suddenly and the occupant 600 from being unable to awake clearly.

At time t1, the illuminance reaches the maximum illuminance. When the maximum illuminance is reached, the control device 400 controls the lighting devices 10 to 30 such that the maximum illuminance is maintained until time t2. As the maximum illuminance, at least 100 lx can be adopted.

As described above, according to the present embodiment, since the long lighting device 10 is disposed so as to at least partly overlap the movable range of the rear seat 520 as viewed from above, it is possible to irradiate the occupant 600 with the awakening light from directly above and to awaken the occupant 600.

Furthermore, in the present embodiment, since the occupant 600 is irradiated with the awakening light from the lighting device 20, the occupant 600 can be more reliably awakened. Furthermore, in the present embodiment, since the occupant 600 is irradiated with the indirect illumination light from the lighting device 30 as the awakening light, the occupant 600 can be more naturally awakened.

Modifications set forth below can be adopted in the present disclosure.

### First Modification

In the present disclosure, an acoustic device 700 may be provided in the moving body 500 instead of or in addition to the lighting device 10.

FIG. 15 is a view of the vehicle interior of the moving body 500 as viewed from the left direction to the right direction in a first modification. As illustrated in FIG. 15, the acoustic device 700 is disposed in the ceiling 510 of the moving body 500 similarly to the lighting device 10.

The acoustic device 700 may be disposed at any position as long as it is in the ceiling 510. The acoustic device 700 is disposed, for example, on either of extension lines in a longitudinal direction of the long lighting device 10 (first lighting device). The acoustic device 700 is disposed so as to at least partly overlap a seating position of an occupant (the movable range of the first seat) when viewed from above. A seating position of the occupant 600 is the movable range of the rear seat 520 (first seat). In the example of FIG. 15, the acoustic device 700 is disposed in front of the lighting device 10.

In a case where the plurality of lighting devices 10 are provided in the ceiling 510 as illustrated in FIG. 3, at least one lighting device 10 among the plurality of lighting devices 10 may be replaced with the acoustic device 700 as long as the illuminance with respect to a front surface of a headrest portion of the rear seat 520 is at least 100 lx. In this case, the acoustic device 700 and the lighting device 10 may be disposed in the order of the acoustic device 700, the lighting device 10, and the lighting device 10 from the front side.

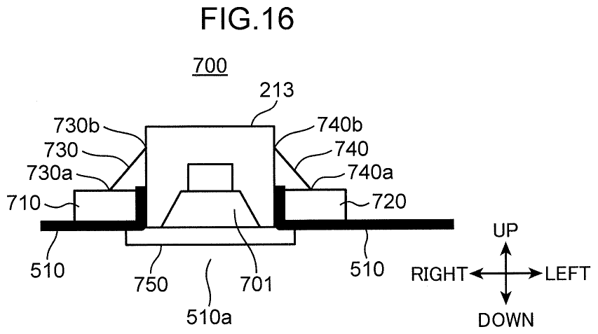

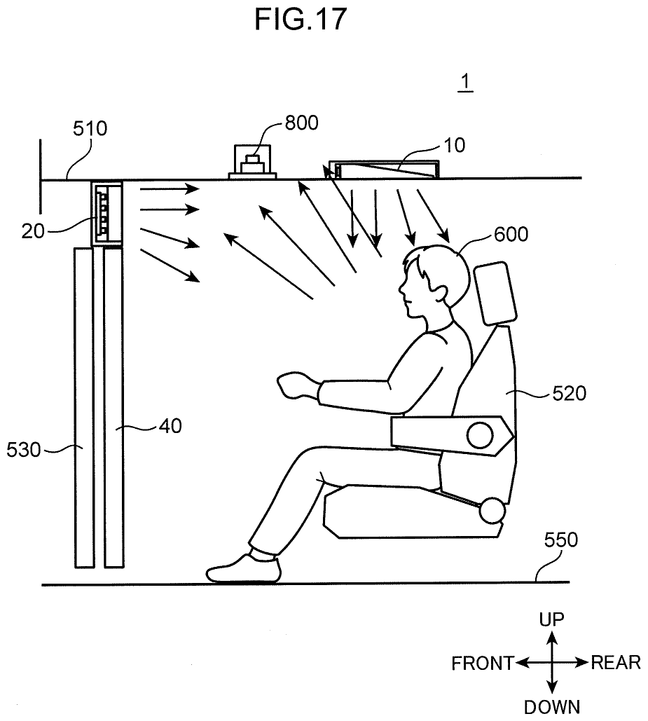

FIG. 16 is a view illustrating an attaching example of the acoustic device 700. In the attaching example illustrated in FIG. 16, the light source 211 and the light guide body 212 of the lighting device 10a illustrated in FIG. 6 are replaced with the acoustic device 700. In detail, a speaker 701 of the acoustic device 700 is disposed in the housing 213. The speaker 701 is disposed in the housing 213 such that a sound output direction faces downward. The attaching example of FIG. 16 is the same as the attaching example of FIG. 6 except that the light source 211 and the light guide body 212 are replaced with the speaker 701.

The acoustic device 700 needs to reduce an influence of heat from the outside (ceiling) of the vehicle. Therefore, in the acoustic device 700, the speaker 701 is stored in the housing 213.

An acoustic cover 750 is attached to the attachment port 510a of the housing 213. The acoustic cover 750 protrudes downward from the ceiling 510. Since the acoustic cover 750 is thin, there is no problem in this protrusion.

As described above, in the attaching example illustrated in FIG. 16, similarly to the attaching example of FIG. 6, the acoustic device 700 can be attached to the ceiling 510 without being screwed, and the designability in the vehicle interior can be enhanced. In addition, in the attaching example illustrated in FIG. 16, it is possible to prevent the body of the occupant 600 from colliding with the acoustic device 700, and it is possible to enhance safety of the occupant 600 in the vehicle interior. In addition, in the attaching example illustrated in FIG. 16, since the free ends 730a and 740a contact the reinforcing members 710 and 720 and do not directly contact the ceiling 510, the ceiling 510 can be protected.

Note that the acoustic cover 750 may be provided so as not to protrude downward from the ceiling 510. For example, a net may be directly attached to the attachment port 510a.

### Second Modification

In the present disclosure, a sound pickup device 800 may be provided in the moving body 500 instead of or in addition to the lighting device 10. Note that the sound pickup device 800 may be provided in the moving body 500 in addition to the lighting device 10 and to the acoustic device 700.

FIG. 17 is a view of the vehicle interior of the moving body 500 as viewed from the left direction to the right direction in a second modification. As illustrated in FIG. 17, the sound pickup device 800 is disposed in the ceiling 510 of the moving body 500 similarly to the lighting device 10. The sound pickup device 800 is disposed so as to at least partly overlap the seating position of an occupant (the movable range of the first seat) as viewed from above. A seating position of the occupant 600 is the movable range of the rear seat 520 (first seat). In the example of FIG. 17, the sound pickup device 800 is disposed in front of the lighting device 10.

The sound pickup device 800 is provided for the purpose of picking up speech voice of the occupant 600, for example, in order to control a voice call or an application. Alternatively, the sound pickup device 800 is provided for the purpose of picking up sound made by the occupant 600 in order to estimate a state of a user. The sound pickup device 800 includes a sound pickup microphone. The sound pickup microphone may have, for example, a beamforming function and an AI sound pickup function. The AI sound pickup function is a function for picking up only sound to be picked up without picking up sound output from the acoustic device 700.

Similarly to the acoustic device 700, the sound pickup device 800 may be disposed at any place as long as it is in the ceiling 510. The sound pickup device 800 may be disposed integrally with the acoustic device 700. In other words, both the acoustic device 700 and the sound pickup device 800 may be stored in the same housing 213. The acoustic device 700, the sound pickup device 800, and the lighting device 10 may be disposed at different places in the ceiling 510.

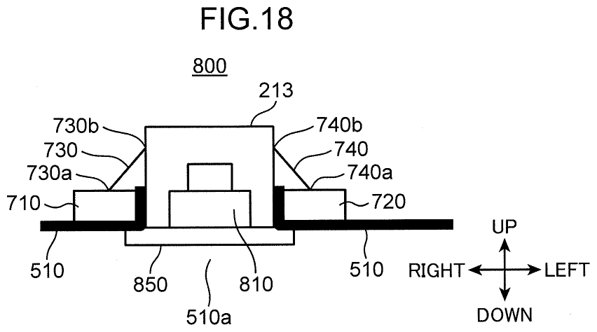

FIG. 18 is a view illustrating an attaching example of the sound pickup device 800. In the attaching example illustrated in FIG. 18, the light source 211 and the light guide body 212 of the lighting device 10a illustrated in FIG. 6 are replaced with the sound pickup device 800. In detail, a sound pickup microphone 810 of the sound pickup device 800 is disposed in the housing 213. The attaching example of FIG. 18 is the same as the attaching example of FIG. 6 except that the light source 211 and the light guide body 212 are replaced with the sound pickup microphone 810.

The sound pickup device 800 needs to reduce an influence of heat from the outside of the vehicle. Therefore, the sound pickup device 800 is stored in the housing 213.

A sound pickup cover 850 is attached to the attachment port 510a of the housing 213. The sound pickup cover 850 protrudes downward from the ceiling 510. Since the sound pickup cover 850 is thin, this protrusion has no problem.

As described above, in the attaching example illustrated in FIG. 18, similarly to the attaching example of FIG. 6, the sound pickup device 800 can be attached to the ceiling 510 without being screwed, and the designability in the vehicle interior can be enhanced. In addition, in the attaching example illustrated in FIG. 18, it is possible to prevent the body of the occupant 600 from colliding with the sound pickup device 800, and it is possible to enhance safety of the occupant 600 in the vehicle interior. In addition, in the attaching example illustrated in FIG. 18, since the free ends 730a and 740a contact the reinforcing members 710 and 720 and do not directly contact the ceiling 510, the ceiling 510 can be protected.

### Third Modification

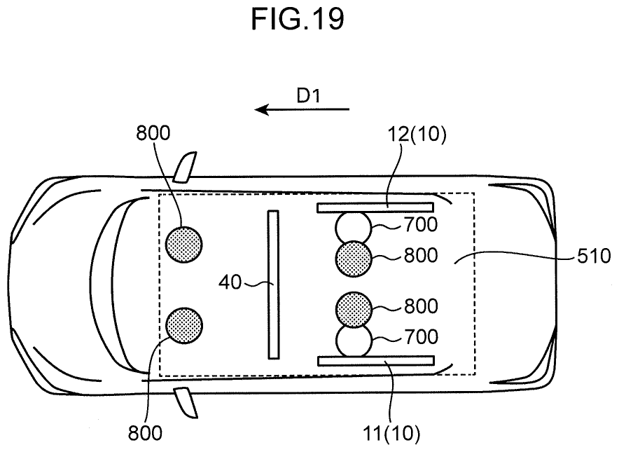

FIG. 19 is a top view of the moving body 500 in which an environment forming system is disposed according to a third modification.

As illustrated in FIG. 19, a pair of the sound pickup devices 800 is disposed on the front seat side of the ceiling 510. The lighting device 10, the acoustic device 700, and the sound pickup device 800 are disposed on the rear seat side of the ceiling 510. In detail, four sound pickup devices 800 are provided above a left front seat, a right front seat, a left rear seat, and a right rear seat, respectively. Two acoustic devices 700 are disposed above the left rear seat and the right rear seat, respectively. On a left end side of the ceiling 510, the lighting device 11 is disposed in parallel to the traveling direction D1, and on a right end side of the ceiling 510, the lighting device 12 is disposed in parallel to the traveling direction D1.

For example, the sound pickup device 800 in the front seat may use the AI sound pickup function to pick up sound of only the occupant 600 seated on the front seat without picking up sound of the acoustic device 700. Similarly, the sound pickup device 800 in the rear seat may use the AI sound pickup function to pick up the sound of only the occupant 600 seated on the rear seat without picking up the sound of the acoustic device 700.

The speaker 701 of the acoustic device 700 is provided in the housing 213, the sound pickup microphone 810 of the sound pickup device 800 is provided in the housing 213, and the light source 211 and the light guide body 212 of the lighting device 10 are provided in the housing 213. The speaker 701, the sound pickup microphone 810, the light source 211, and the light guide body 212 provided in the housing 213 in this manner may be configured to be replaceable.

In this case, the housing 213 serves as a socket. With the replaceable configuration, the acoustic device 700, the sound pickup device 800, and the lighting device 10 can be newly provided without providing the additional attachment port 510a, or a type of the device can be easily changed according to a situation.

In the first to third modifications, instead of or in addition to the acoustic device 700 and the sound pickup device 800, an environment forming device such as a scent releasing device or an air cleaning device may be provided.

### Fourth Modification

As long as the lighting device 10 is disposed so as to at least partly overlap the movable range of the rear seat 520 when viewed from above the moving body 500, the lighting device 10 may be disposed in a direction (left-right direction) perpendicular to the traveling direction D1 of the moving body 500. In this case, more preferably, the lighting device 10 is disposed in front of the headrest 520c when the rear seat 520 is slid to the foremost side and the back portion 520a is raised most. As a result, even when a plurality of occupants are simultaneously seated on the rear seat 520, it is possible to irradiate all the occupants with the awakening light only by at least one lighting device 10.

### Fifth Modification

The lighting device 10 may include the lighting devices 11 and 12 disposed in parallel and a lighting device disposed to be orthogonal to the lighting devices 11 and 12 (in the left-right direction). In addition, the lighting devices 11 and 12 and the lighting device disposed in the left-right direction may be configured to be continuous in a “C” shape. Here, a direction of light distribution of the lighting device installed in the left-right direction may be set to be the longitudinal direction, i.e., the direction of the right side (or left side or both sides) as in the embodiment as long as the face illuminance is 100 lx or more. By disposing the lighting device 10 and setting the light distribution in this manner, the awakening light can be reliably provided to the occupant, for example, even when the occupant is sleeping while facing sideways. In addition, a plurality of lighting devices having a light distribution from the front side toward the rear side of the moving body 500 may be disposed side by side.

### Sixth Modification

The lighting device 20 may not be installed on the wall surface 530 as long as the lighting device 20 is disposed so as to be opposed to the front of the rear seat 520. For example, the lighting device 20 may be installed so as to be suspended from the ceiling 510. In addition, the lighting device 20 may have a support or a leg, and may be installed so as to extend from a side surface in the vehicle interior or from a floor surface in the vehicle interior. Moreover, the lighting device 20 may be attached to the display device 40. In these cases, the lighting device 20 is preferably installed at a height and a position that do not interfere when the occupant gets on and off.

### Seventh Modification

The number of the lighting devices 20 is not limited to one, and may be plural. For example, the plurality of lighting devices 20 may be installed on a back of a headrest of one or more front seats 540, respectively. Alternatively, the plurality of lighting devices 20 may be installed on the back of one or more front seats 540, respectively. In these cases, the lighting device 20 may be installed so as to be embedded in the front seat 540.

### Eighth Modification

The fixture member 1311 is not necessarily provided, and other configurations may be adopted as long as an indirect illumination light can be emitted to the occupant. For example, the lighting device 31 may be provided in front of (on the front seat side of) the display device 40, and an end portion of the display device 40 may also serve as a fixture member. The same applies to the fixture member 1312.

The present disclosure is useful in the technical field of in-vehicle devices.

1. A lighting system comprising at least one first lighting device that is installed in a ceiling in a vehicle interior of a moving body and emits awakening light,
wherein the at least one first lighting device
is a long lighting device including at least one light source, and
is disposed so as to at least partly overlap a movable range of a first seat provided in the vehicle interior when viewed from above.

2. The lighting system according to claim 1, wherein
the at least one first lighting device includes a long light guide body, and
the at least one light source is provided to have a light emitting surface facing a longitudinal direction of the light guide body at one end portion in the longitudinal direction of the light guide body.

3. The lighting system according to claim 1, further comprising a second lighting device,
the second lighting device being disposed to be opposed to a front of the first seat.

4. The lighting system according to claim 3, wherein
the first seat is a rear seat, and
the second lighting device is installed between a front seat and the rear seat.

5. The lighting system according to claim 1, wherein
the at least one first lighting device has variable illuminance,
the lighting system further comprising a control unit that changes the illuminance of the at least one first lighting device over time in an awakening phase of awakening an occupant.

6. The lighting system according to claim 3, wherein
the second lighting device has variable illuminance,
the lighting system further comprising a control unit that changes the illuminance of the second lighting device over time.

7. The lighting system according to claim 1, wherein
the ceiling includes an attachment port to which each of the at least one first lighting device is attached and a reinforcing member provided along the attachment port on a back surface of the ceiling,
each of the at least one first lighting device includes:
a housing embedded in the attachment port; and
a first cantilever spring connected to one side surface in a longitudinal direction of the housing and a second cantilever spring connected to the other side surface in the longitudinal direction of the housing, and
free ends of the first and second cantilever springs are brought into contact with the reinforcing member.

8. The lighting system according to claim 1, wherein in the at least one first lighting device, luminance of the at least one light source is set such that illuminance with respect to a front surface of a headrest portion of the first seat is at least 100 lx.

9. The lighting system according to claim 5, wherein the control unit increases the illuminance of the at least one first lighting device over time from a turn-off state to a maximum illuminance in the awakening phase.

10. The lighting system according to claim 1, further comprising at least one third lighting device,
the at least one third lighting device being an indirect lighting device installed between a front seat and a rear seat.

11. The lighting system according to claim 1, further comprising an acoustic device installed in the ceiling,
the acoustic device being disposed so as to at least partly overlap the movable range of the first seat when viewed from above.

12. The lighting system according to claim 1, further comprising a sound pickup device installed in the ceiling,
the sound pickup device being disposed so as to at least partly overlap the movable range of the first seat when viewed from above.
